(19) United States
(12) Patent Application Publication
(10) Pub. No.: US 2024/0029857 A1
(43) Pub. Date: Jan. 25, 2024

(54) METHOD AND SYSTEM FOR AUTOMATED ACTIVITY RECOMMENDATION IN DIABETES TREATMENT PLANS

(71) Applicant: Roche Diabetes Care, Inc., Indianapolis, IN (US)

(72) Inventors: Jennifer Fisher, Fishers, IN (US); Paul Galley, Cumberland, IN (US); Mark Mears, Westfield, IN (US)

(73) Assignee: Roche Diabetes Care, Inc., Indianapolis, IN (US)

(21) Appl. No.: 18/479,855

(22) Filed: Oct. 3, 2023

Related U.S. Application Data
(63) Continuation of application No. PCT/US2022/026876, filed on Apr. 29, 2022.
(60) Provisional application No. 63/181,863, filed on Apr. 29, 2021.

(51) Int. Cl. G16H 20/30; A61B 5/145; G16H 20/60; G16H 10/20; G16H 50/50; A61B 5/00

(52) U.S. Cl. CPC G16H 20/30; A61B 5/14532; G16H 20/60; G16H 10/20; G16H 50/50; A61B 5/4869

(57) ABSTRACT

A method for generating activity recommendations in a diabetes treatment plan includes receiving physiological data, preferences, and suggested activities for a person with diabetes (PwD), generating physiological profiles for the PwD, providing the physiological profiles to a virtual physiological model, receiving projections from a virtual physiological model, generating weighted values based on the suggested activities and preference data, each weighted value corresponding to a likelihood of the PwD adhering to a suggested activity, ranking each activity based on the estimated change in the physiological characteristic of a projection associated with the activity relative to a baseline physiological projection and scaled by the weighted value corresponding to each activity, and generating an output including a predetermined number of suggested activities ordered based on the ranking of activities that provide a greatest change in the physiological characteristic given the likelihood of the PwD adhering to the suggested activities.

### CLAIM OF PRIORITY

This application is a continuation of PCT/US2022/026876, filed Apr. 29, 2022, which claims priority to U.S. Provisional Application No. 63/181,863, filed on Apr. 29, 2021, the entire contents of which are hereby incorporated herein by reference.

### TECHNICAL FIELD

The disclosure relates generally to the field of treatment for persons with diabetes and, more specifically, to systems that assist in selecting activity goals in a treatment plan for the person with diabetes.

### BACKGROUND

Diabetes mellitus, which is commonly referred to as diabetes, is a category of chronic disorders that reduces or eliminates the ability of the human body to metabolize dietary glucose due to an insufficient ability of the pancreas to produce the hormone insulin, a resistance to insulin, or a combination of insulin insufficiency and insulin resistance. In particular, the onset of type 2 diabetes often occurs in patients whose bodies still produce some level of insulin but who have developed a degree of resistance to insulin, which leads to elevated levels of blood glucose that can lead to ketoacidosis and other comorbidities if left untreated. For explanatory purposes, references to type 2 diabetes also include the condition known as “prediabetes,” which is a form of mild insulin resistance that causes an elevation in average blood glucose levels that may progress to type 2 diabetes. Some persons with diabetes (PwDs), and type 2 diabetes in particular, can make changes to diet, exercise, and sleep hygiene regimens that slow or sometimes reverse the progress of diabetes. For example, proper management of type 2 diabetes enables some PwDs to delay or prevent the need to receive external insulin, the need to take other diabetic medications, and to reduce the likelihood of diabetic comorbidities. Improvements to diet, exercise, sleep hygiene, and medication adherence can also be beneficial to PwDs who are dependent upon external insulin or other diabetes related medications in avoiding the need to increase doses of insulin due to ongoing insulin resistance and in reducing the likelihood of diabetic comorbidities.

While diabetes treatment plans that incorporate improvements to diet, exercise, sleep hygiene, and medication adherence provide benefits to PwDs that are well known to the art, many challenges remain in having PwDs implement these plans consistently over time for effective diabetes management. Diabetes coaches are professionals who provide advice and planning to help PwDs commit and adhere to these plans, which often includes setting goals for achieving improvements in one or more areas of diet, exercise, sleep hygiene, and medication adherence for PwDs who are prescribed medications. While coaches provide valuable assistance to PwDs, in practice the coaches face several hurdles in devising diabetes treatment plans with goals for PwDs that provide benefits to the PwD and that the PwD can adhere to on a consistent basis. Coaches often see a large number of PwDs in comparatively short coaching sessions and may not have sufficient time and resources to provide highly individualized plans to each PwD. These constraints often lead to a “one size fits all” approach to providing plans to many PwDs, and especially PwDs who do not have extensive experience in managing diabetes. Such a plan may not provide optimal results to each PwD and some PwDs may not adhere to the plan even if following the plan would provide benefits to treating diabetes. Given these challenges, improvements to technology that provide customized goal recommendations for each PwD would be beneficial.

### SUMMARY

In one embodiment, a method for generating activity recommendations in a diabetes treatment plan includes receiving, with a processor, physiological data for a person with diabetes (PwD), preference data for the PwD, and a plurality of suggested activities for the PwD, and generating, with the processor, a plurality of physiological profiles for the PwD. The plurality of physiological profiles include a baseline physiological profile based on the physiological data for the PwD, and a plurality of activity physiological profiles, each activity physiological profile corresponding to one activity in the plurality of suggested activities, and each activity physiological profile being based on the physiological data for the PwD and a modification of the physiological data associated with the one activity in the plurality of suggested activities corresponding to the activity physiological profile. The method further includes providing, with the processor, the plurality of physiological profiles to a virtual physiological model, receiving, with the processor, a plurality of projections for the PwD from the virtual physiological model, each projection in the plurality of projections providing an estimated change in a physiological characteristic in the PwD during a predetermined time period corresponding to one physiological profile in the plurality of physiological profiles, generating, with the processor, a plurality of weighted values based on the plurality of suggested activities and the preference data, each weighted value corresponding to a likelihood of the PwD adhering to a corresponding one of the suggested activities, ranking, with the processor, each activity in the plurality of suggested activities based on the estimated change in the physiological characteristic of a projection in the plurality of projections associated with the activity relative to a baseline physiological projection in the plurality of projections corresponding to the baseline physiological profile and scaled by the weighted value corresponding to each activity, and generating, with the processor, an output including a predetermined number of the plurality of suggested activities in order based on the ranking to identify one or more suggested activities that provide a greatest change in the physiological characteristic given the likelihood of the PwD adhering to the suggested activities.

In another embodiment, a system for generating activity recommendations has been developed. The system includes a memory, a network interface device, and a processor operatively connected to the memory and the network interface device. The processor is configured to store physiological data for a person with diabetes (PwD), preference data for the PwD, and a plurality of suggested activities for the PwD in the memory, and generate a plurality of physiological profiles for the PwD. The plurality of physiological profiles include a baseline physiological profile based on the physiological data for the PwD, and a plurality of activity physiological profiles, each activity physiological profile corresponding to one activity in the plurality of suggested activities, and each activity physiological profile being based on the physiological data for the PwD and a modification of the physiological data associated with the one activity in the plurality of suggested activities corresponding to the activity physiological profile. The processor is further configured to transmit, with the network interface device, the plurality of physiological profiles to a virtual physiological model service, receive, with the network interface device, a plurality of projections for the PwD from the virtual physiological model service, each projection in the plurality of projections providing an estimated change in a physiological characteristic in the PwD during a predetermined time period corresponding to one physiological profile in the plurality of physiological profiles, generate a plurality of weighted values based on the plurality of suggested activities and the preference data, each weighted value corresponding to a likelihood of the PwD adhering to a corresponding one of the suggested activities, rank each activity in the plurality of suggested activities based on the estimated change in the physiological characteristic of a projection in the plurality of projections associated with the activity relative to a baseline physiological projection in the plurality of projections corresponding to the baseline physiological profile and scaled by the weighted value corresponding to each activity, and generate an output including a predetermined number of the plurality of suggested activities in order based on the rank to identify one or more suggested activities that provide a greatest change in the physiological characteristic given the likelihood of the PwD adhering to the suggested activities.

### BRIEF DESCRIPTION OF THE DRAWINGS

The advantages, effects, features and objects other than those set forth above will become more readily apparent when consideration is given to the detailed description below. Such detailed description makes reference to the following drawings, wherein:

FIG. 1 is a diagram of a system that provides automated generation of suggested activities that reduce average blood glucose or body mass in a person with diabetes based on projections of a virtual physiological model and preference data of the person with diabetes.

FIG. 2 is a block diagram of a process for generation of suggested activities that provide a change in a physiological characteristic, such as reducing average blood glucose or body mass, in a person with diabetes based on projections from a virtual physiological model and preference data of the person with diabetes.

### DETAILED DESCRIPTION

These and other advantages, effects, features and objects are better understood from the following description. In the description, reference is made to the accompanying drawings, which form a part hereof and in which there is shown by way of illustration, not limitation, embodiments of the inventive concept. Corresponding reference numbers indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, and features falling within the spirit and scope thereof as defined by the embodiments described herein and the embodiments below. Reference should therefore be made to the embodiments described herein and embodiments below for interpreting the scope of the inventive concept. As such, it should be noted that the embodiments described herein may have advantages, effects, and features useful in solving other problems.

The devices, systems and methods now will be described more fully hereinafter with reference to the accompanying drawings, in which some, but not all embodiments of the inventive concept are shown. Indeed, the devices, systems and methods may be embodied in many different forms and should not be construed as limited to the embodiments set forth herein; rather, these embodiments are provided so that this disclosure will satisfy applicable legal requirements.

Likewise, many modifications and other embodiments of the devices, systems and methods described herein will come to mind to one of skill in the art to which the disclosure pertains having the benefit of the teachings presented in the foregoing descriptions and the associated drawings. Therefore, it is to be understood that the devices, systems and methods are not to be limited to the specific embodiments disclosed and that modifications and other embodiments are intended to be included within the scope of the embodiments. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

The description herein references computer systems that employ various components including, but not limited to, processors, memories, and network interfaces. As used herein, the term “processor” refers to one or more digital logic devices that execute stored program instructions to implement digital logic operations in a computing system. Examples of processors include digital logic devices that implement one or more central processing units (CPUs), graphics processing units (GPUs), neural network processors (NPUs), digital signal processors (DSPs), field programmable gate arrays (FPGAs), application specific integrated circuits (ASICs), and any other suitable digital logic devices in an integrated device or as a combination of devices that operate together to implement the processor. During operation, each processor executes stored program instructions and accesses data that are stored in a memory. As used herein, the term memory refers to both non-volatile and volatile data storage devices. Non-volatile data storage devices include magnetic disks, optical disks, solid state NAND and phase change memory devices, and any other suitable data storage device that does not require active electrical power to maintain the state of stored data. Volatile data storage devices refer static and dynamic random access memory (RAM) and any other data storage devices that store data while receiving an active electrical power supply to maintain the state of the stored data. As used herein, the term “network” refers to any communication system that enables two or more computing systems to send and receive data during operation, where common examples include local area networks (LANs) and wide area networks (WANs), including the Internet. Each computing system accesses the network using one or more network interface devices that the corresponding processor uses to send and receive data, where common examples of network interface devices include Ethernet network interface cards for wired network connectivity or wireless local area network (WLAN) or wireless wide area network (WWAN) devices for wireless network connectivity.

The description below refers to a diabetes coach, or more simply a “coach”. A diabetes coach is any person who is qualified to provide advice to a person with diabetes regarding changes in daily habits such as diet, exercise, sleep hygiene, or medication adherence for PwDs who are prescribed medication. The diabetes coach is not necessarily a healthcare provider such as a doctor, nurse, or certified diabetes educator (CDE), although any of these professionals may act as a diabetes coach. The illustrative examples of the coach and person with diabetes provide context to the operation of the embodiments described herein.

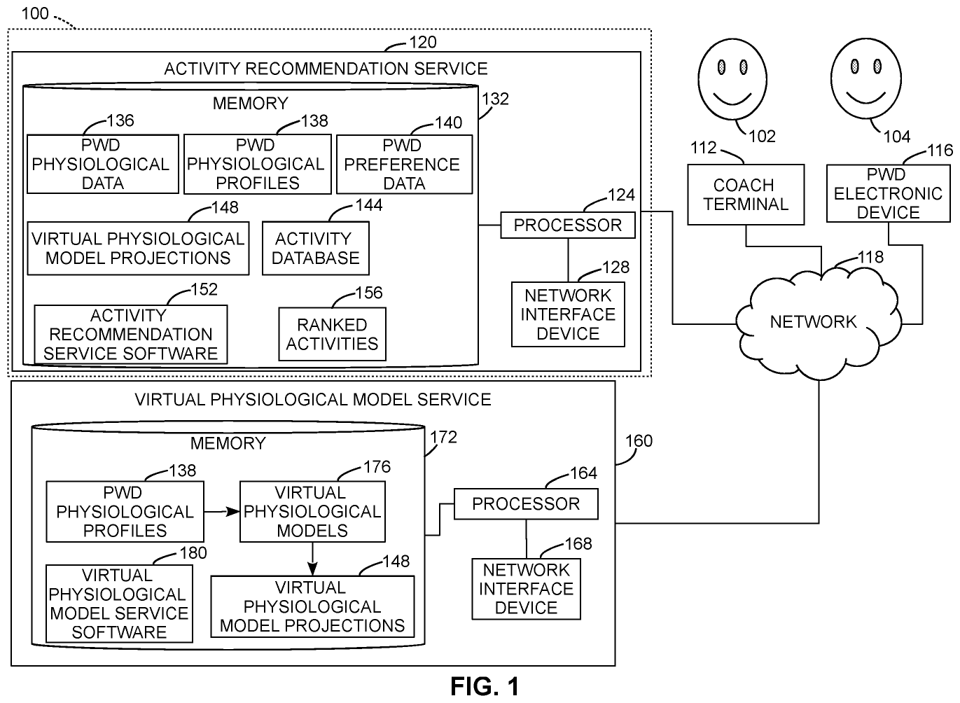

FIG. 1 depicts a system 100 that automatically generates one or more activity recommendations for a person with diabetes (PwD) 104 based on physiological data and preference data for the PwD 104. For illustrative purposes, the PwD 104 is assumed to be diagnosed with either type 2 diabetes or with prediabetes (referred to jointly as “diabetes” below) that the PwD can manage by meeting a goal to perform one or more activities. As used herein, the term “activity” refers to a modification of diet, exercise, sleep hygiene, or medication adherence for PwDs who are prescribed medications to improve at least one physiological characteristic related to diabetes. Two non-limiting examples of physiological characteristics include achieving desirable blood glucose levels or body mass levels for the PwD. A goal refers to a recommended activity that the PwD sets as a goal to perform in a consistent manner to realize the improved physiological characteristic. In the configuration of FIG. 1, a diabetes coach 102 and the PwD 104 use the system 100 to generate the one or more activity recommendations for the PwD 104. In the embodiment of FIG. 1, the system 100 includes an activity recommendation service 120. The activity recommendation service 120 is communicatively connected to a virtual physiological model service 160, and in alternative embodiments the system 100 incorporates both the activity recommendation service 120 and the virtual physiological model service 160. The coach uses a terminal 112 to access the system 100, and the PwD 104 uses an electronic device 116 to provide input data to the system 100 and optionally to receive activity recommendations from the system 100 directly or to communicate with the coach 102 via the terminal 112. The activity recommendation service 120, virtual physiological model service 160, terminal 112, and electronic device 116 use a network 118 for communication.

In the system 100, the terminal 112 is, for example, a personal computer (PC), tablet computing device, smartphone, or other suitable computing device that implements client software to enable the coach 102 to access the system 100 and, in some embodiments, to communicate with the PwD electronic device 116. The PwD electronic device 116 is another PC, tablet computing device, smartphone, or other suitable computing device that is typically owned by or available to the PwD 104. The PwD electronic device 116 implements client software that enables the PwD 104 to provide answers to diagnostic questions to provide at least a portion of the relevant physiological data for the PwD 104 to the activity recommendation service 120 in the system 100. The PwD electronic device 116 also enables the PwD 104 to provide preference data to the activity recommendation service 120. An example of a client software program in the terminal 112 and the PwD electronic device 116 is a commercially available web browser that acts as a client to one or more web services that the activity recommendation service 120 provides to enable the terminal 112 and the PwD electronic device 116 to act as user interfaces to the system 100. In some configurations, the terminal 112 and the PwD electronic device 116 further include audio or audio/visual devices that enable direct communication between the coach 102 and the PwD 104 to conduct a remote coaching session, although the coaching session may also occur in-person.

In the system 100, the activity recommendation service 120 is a computing system that further includes a processor 124, network interface device 128, and memory 132. The activity recommendation service 120 receives physiological and preference data corresponding to the PwD. The activity recommendation service 120 further receives projections from the virtual physiological model service 160 that estimate changes in at least one physiological characteristic of the PwD 104 in response to suggested activities. The activity recommendation service 120 generates a ranked output of suggested activities based on both the projections from the virtual physiological model service 160 and the preference data. In the activity recommendation service 120, the memory 132 stores PwD physiological data 136, PwD physiological profiles 138, PwD preference data 140, an activity database 144, virtual physiological model projections 148, stored program instructions for activity recommendation service software 152, and an output of ranked activities 156.

In the memory 132, the physiological data 136 are medical data including age, sex, height, weight, average blood glucose levels, sleep schedules, metabolic data including diet and exercise, current medication data, diagnosed medical conditions other than diabetes, and any other medically relevant parameters of the PwD 104 that the activity recommendation service 120 uses as some or all of the input data to generate PwD physiological profiles 138. As described in further detail below, each PwD physiological profile 138 includes all or a portion of the physiological data 136 that are required as inputs to a virtual physiological model 176 in the virtual physiological model service 160. In some configurations, the system 100 receives all or a portion of the PwD physiological data 136 from an external electronic medical record (EMR) system via the network 118, a data storage device, or from entry via the coach terminal 112 or PwD electronic device 116. One of the PwD physiological profiles 138 is referred to as a “baseline physiological profile”, which the activity recommendation service 120 generates based only the actual physiological data 136 of the PwD 104 to represent the current physiology and activities of the PwD 104 at the time of the coaching session. The other PwD physiological profiles 138 are also referred to as “activity physiological profiles”. Each activity physiological profile incorporates both the PwD physiological data 136 and modifications to the physiological data of the PwD 104 that occur in response to the PwD 104 performing one of the suggested activities from the activity database 144. As described in further detail below, the activity recommendation service 120 identifies the effects of a suggested activity to change the physiological parameters of the PwD 104 in each PwD physiological profile 138 based on data associated with each activity in the activity database 144. During operation, the activity recommendation service 120 transmits the PwD physiological profiles 138 to the virtual physiological model service 160, which uses the PwD physiological profiles 138 as inputs to a virtual physiological model 176 that generates projections to estimate changes in physiological characteristics of the PwD 104 corresponding to the physiological data in each of the physiological profiles 138.

In the memory 132, the PwD preference data 140 include information that the PwD 104 provides concerning the types of activities that the PwD prefers to perform to manage diabetes. In one configuration, the PwD 104 provides numeric data in the survey answers to predetermined questions that gauge preferences for performing different activities using a numeric range, such as a 1 to 10 scale or other suitable scale. The numeric data enable the activity recommendation service 120 to quantify preferences for the PwD 104. In the embodiment of FIG. 1, the PwD 104 submits answers to a survey using the PwD electronic device 116, and the PwD electronic device 116 transmits the survey question answers to the activity recommendation service 120 prior to a meeting with the coach 102. In another configuration, the coach 102 elicits responses to the survey questions from the PwD 104 during a coaching session and enters the responses using the terminal 112. In some configurations, the preference data include additional implicit preference data pertaining to the PwD 104 that provide geographic data corresponding to a home location of the person with diabetes, and demographic information of the person with diabetes. The geographic data influence the applicability of different types of activities for the PwD. For example, an indoor exercise activity may be more suitable to a PwD who lives in an urban or cold-weather climate while outdoor activities may be more suited to a PwD who lives in a rural setting or one with warmer weather. The PwD 104 provides other demographic information such as income, working schedule, educational level, transportation access, and similar parameters as implicit preference data used in identification of the preferences of the PwD 104 to various recommended activities. For example, a PwD 104 who does not own a motor vehicle may have access to activities that are only located within a comparatively short distance from home relative to a PwD who does have access to the motor vehicle. In another example, a PwD who works at night may benefit from specific sleep hygiene activities that are less critical for a PwD with a diurnal sleep pattern.

In the memory 132, the activity database 144 includes a predetermined set of diet, exercise, sleep hygiene, and medication adherence activities that are each linked to metabolic characteristics of the PwD. For example, a diet activity corresponds to a change in caloric intake, and an exercise activity relates to a change in caloric consumption. The activity database 144 also stores one or more metabolic characteristics related to sleep hygiene activities since improved sleep hygiene can increase the metabolism of calories directly, lead to reduced caloric intake by reducing over-eating due to sleep deprivation, and provide a boost to energy levels that enable the PwD to perform other activities such as exercise. The activity database 144 also stores medication adherence activities that apply to PwDs who are taking medications that affect diabetes either directly or indirectly, and the physiological affects of increased medication adherence may include a direct reduction in average blood glucose levels or other improvements to metabolism. During operation, the activity recommendation service 120 identifies a change in metabolism that occurs in response to a selected activity based on the data stored in the activity database 144. For example, a diet activity recommendation to reduce the consumption of a soft drink, with a number of calories (e.g. 130 calories) in reduced caloric consumption if the PwD 104 substitutes water or unsweetened tea for the soft drink. Other activities may increase caloric intake but with foods that are less likely to have harmful effects to the PwD 104, such as a recommendation to consume nuts (e.g. 200 calories) as a replacement for another food item that may include fewer calories but that has a higher proportion of carbohydrate calories compared to protein or fat calories. The activity database 144 stores caloric contents of various foods and drinks from publicly available nutrition databases in association with each dietary activity, and the activity recommendation service 120 calculates a change in calories, consumed carbohydrates, fat, and protein, or other dietary information for performing the activity compared to the baseline caloric information that is included in the PwD physiological data 136. The activity database 144 also stores base caloric information pertaining to the number of calories that are metabolized during different exercise activities and different intensity levels of each activity, such as base caloric metabolism data for walking at 3 miles per hour compared to running at 7 miles per hour. The activity recommendation service 120 calculates the final estimate of burned calories based on the selected activity, the body mass of the PwD 104, and the intended duration of the activity. The number of metabolized calories for an activity increases with body mass, intensity, and duration for any given activity. In some cases, the activity recommendation service 120 also identifies the change in caloric consumption or caloric metabolism based on how often the PwD 104 performs a recommended activity, such as daily activities or activities that are performed one or more times weekly.

The memory 132 also stores the virtual physiological model projections 148 and the activity recommendation service software 152. The virtual physiological model projections 148 provide estimates of changes in a physiological characteristic for the PwD 104 if the PwD 104 performs one or more of the suggested activities in the activity database 144. As described in further detail below, the virtual physiological model service 160 generates and transmits the physiological model projections 148 to the activity recommendation service 120. The activity recommendation service software 152 includes any stored program instructions that the processor 124 executes to perform the operations of the activity recommendation service 120 that are described herein. During operation, the processor 124 executes the activity recommendation service software 152 to perform a ranking algorithm of suggested activities from the activity database 144 based on the estimated change in physiological characteristic from the physiological model projections 148 scaled by the PwD preference data 140 to generate the ranked activities 156 as an output that is provided to the coach terminal 112 or PwD electronic device 116. The ranked activities 156 are ordered based on the activities with the greatest estimated benefit and likelihood of adherence for the PwD 104. The activity recommendation service software 152 also provides a communication interface with the virtual physiological model service 160 via the network 118 to enable transmission of the PwD physiological data 136 to the virtual physiological model service 160 and to receive the virtual physiological model projections 148. The activity recommendation service software 152 further implements a user interface to receive input and provide output data to either or both of the coach terminal 112 and the PwD electronic device 116. In the configuration of FIG. 1, the system 100 includes a networked activity recommendation service 120 that implements a remote user interface, such as a web server or other suitable server program, to permit access from either or both of the coach terminal 112 and PwD electronic device 116 using a web browser or other suitable client software program that communicates with the system 100 via the network 118.

In the embodiment of FIG. 1, the virtual physiological model service 160 is a computing system that further includes a processor 164, network interface device 168, and memory 172. The virtual physiological model service 160 receives physiological data for the PwD and uses a virtual physiological model 176 to project the effects of a suggested activity that the PwD performs over time in changing at least one physiological characteristic, such as changing average blood glucose levels or body mass. The virtual physiological model service 160 does not process information related to activities directly. Instead, the activity recommendation service 120 generates different physiological profiles 138 for the baseline of the PwD 104 and each suggested activity for the PwD 104. The virtual physiological model service 160 receives different sets of physiological profiles 138 from the activity recommendation service 120 that include different levels of any or all of caloric consumption, caloric metabolism, sleep hygiene, or medication adherence for different activities to generate the projections. Each projection includes an estimate of the change in the at least one physiological characteristic over a predetermined time period given the activity, and the virtual physiological model service 160 also generates a baseline projection with an estimate of the at least one physiological characteristic if the PwD 104 maintains his or her current activities without performing any of the newly suggested activities.

In the virtual physiological model service 160, the memory 172 stores PwD physiological profiles 138, one or more virtual physiological models 176, stored program instructions that implement the virtual physiological model service software 180, and one or more virtual physiological model projections 148. The PwD physiological profiles 138 include the same data that are described above in conjunction with the activity recommendation service 120 and, in the embodiment of FIG. 1, the virtual physiological model service 160 receives the PwD physiological profiles 138 from the activity recommendation service 120 via the network 118.

In the memory 172, the virtual physiological models 176 refer to one or more digital models of the human body that simulate physiological processes in the body of the PwD to generate projections of estimated changes to the physiological characteristics over time. One non-limiting example of a commercially available virtual physiological modeling service that provides a virtual physiological model is the Bodylogical digital twin service that is available from PricewaterhouseCoopers of London, England. During operation, the processor 164 executes the virtual physiological model service software 180 to perform a simulation using different sets of the PwD physiological profiles 138 as inputs to a virtual physiological model 176 to generate the virtual physiological model projections 148. The PwD physiological profiles 138 provide parameter information for the virtual physiological models 176 to use in order to provide projections that are customized for the PwD 104 or for other PwDs who each have different physiological data. During operation, the virtual physiological model service 160 uses the PwD physiological profiles 138 as inputs to the virtual physiological model 176 to generate projections with an estimate of changes to at least one physiological characteristic, such as average blood glucose levels or body mass. Each projection corresponds to an estimated state of the PwD that the virtual physiological model service software 180 generates for a given time horizon, such as an expected change in blood glucose, body mass, or other physiological characteristics over a 30 day, three month, six month, 1 year, or other selected time period. Additionally, the virtual physiological model service 160 uses the virtual physiological models 176 to generate a baseline projection based on the baseline physiological profile. The baseline projection includes an estimate of the at least one physiological characteristics for the PwD 104 based on the existing physiological data assuming that the PwD 104 makes no changes to current activities. In the embodiment of FIG. 1, the virtual physiological model service 160 transmits the generated virtual physiological model projections 148, including the baseline projection and one or more projections that are generated based on different recommended activities, to the activity recommendation service 120. The activity recommendation service 120 uses the baseline projection and the projections for different selected activities to identify the estimated changes in the physiological characteristics relative to the baseline projection to identify the activities with the greatest potential benefit for the PwD 104.

While the system 100 includes the activity recommendation service 120 with the processor 124, those of skill in the art will recognize that the system 100 may be implemented using a single computing system with a single processor or multiple computing systems that incorporate multiple processors. For example, another implementation of the activity recommendation service 120 uses a single computing system or divides the functionality described herein into a greater number of computing systems. Additionally, in many practical embodiments the activity recommendation service 120 is implemented using a cluster of multiple individual computing devices with redundant data storage devices to provide fault tolerance and scalability using clustering techniques that are generally known to the art. In an alternative configuration, the system 100 incorporates the functionality of the activity recommendation service 120 and the virtual physiological model service 160 into a single computing system using a single computing device or cluster of multiple individual computing devices. In FIG. 1, the activity recommendation service 120 stores a copy of the virtual physiological model projections 148 in the memory 132 that the virtual physiological model service 160 generates and transmits to the activity recommendation service 120, but a single memory may store the physiological model projections 148 in an alternative system configuration that combines the activity recommendation service 120 and the virtual physiological model service 160. Furthermore, while the system 100 is shown as a networked service for illustrative purposes, those of skill in the art will recognize that the system 100 could be implemented entirely within the coach terminal 112, the PwD electronic device 116, or another individual computing device. As such, any references to the operations of separate processors that perform portions of the functionality in the system 100 should be understood to be interchangeable with a reference to the operation of a single processor and vice versa.

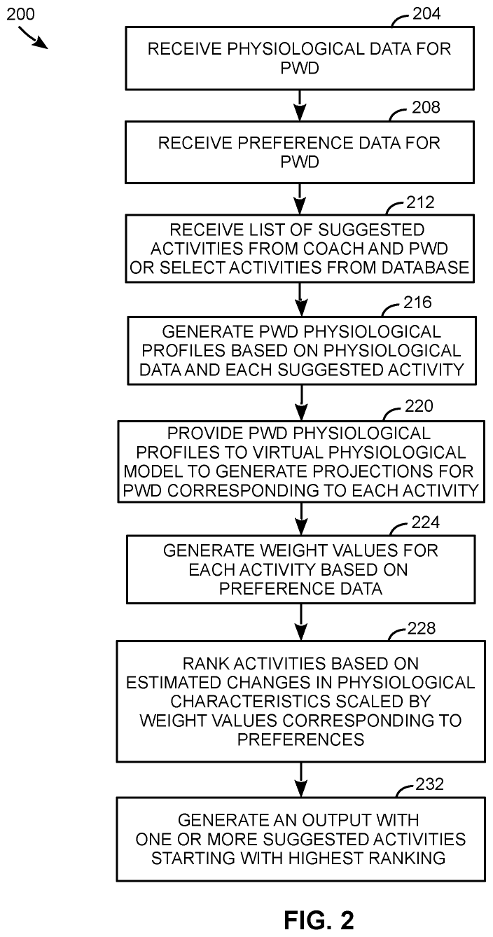

FIG. 2 depicts a process 200 for generation of suggested activities a physiological characteristic, such as reducing average blood glucose or body mass, in a person with diabetes based on projections from a virtual physiological model and preference data of the person with diabetes. In the description below a reference to the process performing a function or action refers to the operation of one or more digital processors to execute stored program instructions to perform the function or action. The process 200 is described in conjunction with the system 100 of FIG. 1 for illustrative purposes.

The process 200 begins as the activity recommendation service 120 receives physiological data for the PwD 104 (block 204) and preference data for the PwD 104 (block 208), and these data may be received in any order or concurrently during the process 200. In one configuration, the system 100 receives the physiological data from an external electronic medical records (EMR) service (not shown) in a using a standardized medical record systems such as the Fast Healthcare Interoperability Resources (FHIR) standard, HL7 standard, or another accepted standard interchange system for medical record data. The activity recommendation service 120 stores the EMR data as the PwD physiological data 136 in the memory 132. While not a requirement, the transfer of EMR data to the activity recommendation service 120 often occurs prior to an initial coaching session, and the activity recommendation service 120 is configured to store the PwD physiological data 136 between coaching sessions to track the history and progress of the PwD 104 over time. Automated transfer of EMR data reduces the need for manual data entry, and the PwD 104 grants consent for the data transfer prior to the system 100 receiving any EMR data to comply with any applicable medical data privacy regulations. The activity recommendation service 120 may receive EMR data for a large number of PwDs to enable the identification and prioritization of PwDs who receive coaching services. In another configuration, the system 100 receives at least some of the physiological data from the PwD 104 directly via the PwD electronic device 116. In this configuration, the PwD 104 accesses a website or other remote user interface that the activity recommendation service 120 provides to elicit specific pieces of physiological data from the PwD 104. The PwD 104 uses the electronic device 116 to enter pieces of physiological information and submits answers to survey questions that provide preference data prior to a coaching session in order to reduce the amount of time required to collect the preliminary physiological data during a coaching session. In another configuration, the coach 102 collects either or both of the physiological data and preference data during a coaching session and the activity recommendation service 120 provides a similar website or remote interface to the coach terminal 112 to receive the physiological data and preference data from the PwD 104. In some configurations, the physiological data 136 include both EMR data and physiological data that are received from the PwD electronic device 116 and coach terminal 112.

The process 200 continues as the system 100 identifies potential activities by receiving a list of suggested activities from the coach 102 and PwD 104, or selecting suggested activities from the activity database 144 (block 212). In one configuration, the coach 102 and the PwD discuss potential activities and the coach 102 enters selected suggested activities into a user interface of the coach terminal 112, where each activity corresponds to one of the activities in the activity database 144. In another configuration, the activity recommendation service 120 selects all of the activities in the activity database 144 or selects activities from categories of activities, such as a diet, exercise, sleep hygiene, and medication adherence categories, to identify potential suggested activities for the PwD instead of or in addition to receiving specific suggested activities from the coach 102 and PwD 104. As described above, the selected activities from the activity database 144 include data corresponding to both a kind of activity and, where appropriate, the intensity and frequency of performing the activity.

During the process 200, the activity recommendation service 120 generates the physiological profiles 138 for the PwD 104 including a baseline physiological profile for the PwD 104 based on the physiological data 136 and activity physiological profiles that each correspond to the PwD 104 performing one of the suggested activities (block 216). To generate the baseline physiological profile, the activity recommendation service 120 processes the physiological data 136 that correspond to the current medical condition of the PwD 104 and that incorporate metabolic data for the current diet and exercise activities, sleep patterns, and medication adherence of the PwD 104.

To generate activity physiological profiles 138 for suggested exercise and diet activities in the activity database 144, the activity recommendation service 120 identifies either an increase in caloric metabolism in response to an exercise activity or a change in consumed calories in response to a diet activity. In some instances, the activity recommendation service 120 further identifies changes in the overall proportions of calories from different macronutrients consumed by a diet such as identifying a decrease in the proportion of carbohydrate calories consumed compared to protein and fat calories. The activity recommendation service 120 also calculates the total caloric consumption of different exercise activities based on the body mass of the PwD 104 and the duration, intensity, frequency, and type of each exercise. Thus, in addition to identifying activities that correspond to different exercise types, the activity recommendation service 120 further identifies different activities that each correspond to a single type of exercise with varying levels of intensity, duration, and frequency that affect the corresponding changes in caloric metabolism. The activity recommendation service 120 generates each activity physiological profile as a modification of the baseline physiological profile including altered parameters that reflect the effects of the suggested activity on the physiology of the PwD 104. Examples of altered physiological parameters in the activity physiological profiles 138 include, for example, changes in total caloric metabolism, resting metabolism for exercise activities, and total caloric consumption along with changes proportions of dietary macronutrients for diet activities.

The activity recommendation service 120 further modifies one or more physiological parameters that are related to sleep hygiene or medication adherence to generate activity physiological profiles 138 that correspond to suggested sleep hygiene and medication adherence activities. A sleep hygiene activity recommendation refers to a suggested change in the duration of sleep, pattern of sleep, or quality of sleep that the PwD 104 should perform to reduce negative metabolic effects of insufficient or low-quality sleep. For example, one sleep hygiene activity suggests that the PwD set a regular bedtime and to wake up time each day to establish a consistent sleep pattern. The activity recommendation service 120 modifies sleep parameters in the baseline physiological profile to include the new sleep hygiene parameters from the activity database 144 to generate the activity physiological profile 138 for a suggested sleep hygiene activity. Medication adherence activities correspond to techniques for the PwD 104 to follow the official on-label directions for taking a given medication in a consistent manner, which is applicable to some PwDs who consume a prescribed diabetes medication. To cite a non-limiting example, at least some forms of Metformin specify prandial consumption, and the activity database 144 stores an activity suggestion for scheduling regular meal times that encourages the habit of taking Metformin with scheduled meals for improved adherence. The activity recommendation service 120 modifies medication adherence parameters in the baseline physiological profile, which include data as to how often the PwD 104 actually uses the medication as indicated on the label, to include the new medication adherence parameters from the activity database 144 to generate the activity physiological profile 138 for a suggested medication adherence activity.

The process 200 continues as the activity recommendation service 120 provides the PwD physiological profiles 138 to the virtual physiological model service 160, which generates projections of changes in at least one physiological characteristic for the PwD 104 over time in each of the PwD physiological profiles 138 using the virtual physiological models 176 (block 220). In the system 100, the activity recommendation service 120 uses the network interface 128 to transmit the PwD physiological profiles 138 for the PwD 104 to the virtual physiological model service 160 via the network 118. The memory 172 of the virtual physiological model service 160 stores the PwD physiological profiles 138 as inputs to the virtual physiological models 176. As described above, the PwD physiological profiles 138 include the baseline profile that the system 100 collects for the PwD 104 using current diet, exercise, sleep hygiene, and medication adherence activities without any modification to the activities for the PwD 104. Each of the other PwD physiological profiles 138 includes modified physiological data corresponding to each activity that include the changes to metabolism or other physiological parameters that occur if the PwD 104 performs one of the suggested activities. The processor 164 in the virtual physiological model service 160 executes the virtual physiological model software 180 to apply each PwD physiological profile 138 to a corresponding virtual physiological model 176 to produce the virtual physiological model projections 148. The virtual physiological model service 160 uses the network interface device 168 to transmit the virtual physiological model projections 148 to the activity recommendation service 120 via the network 118. As depicted in FIG. 1, the corresponding network interface 128 of the activity recommendation service 120 receives the virtual physiological model projections 148 and the processor 124 stores a copy of the virtual physiological model projections 148 in the memory 132.

In more detail, the virtual physiological model service 160 performs a simulation for each PwD physiological profile 138 to generate a baseline projection including estimates of physiological characteristics of the PwD 104 over time for the baseline physiological projection and an activity projection for each of the PwD activity physiological profiles 138. For example, the virtual physiological model service 160 performs a simulation that generates a baseline projection that estimates the HbA1c for the PwD 104 at 6.8% after 6 months given the baseline PwD physiological profile 138 for the PwD 104. However, if the PwD 104 performs a suggested exercise activity, then the virtual physiological model service 160 performs another simulation with the corresponding activity physiological profile containing a different set of physiological parameters with an increased caloric metabolism value due to that activity, which produces a different estimated HbA1c level of, for example, 6.3%. The virtual physiological model service 160 performs a similar simulation based on the different PwD physiological profiles 138 for each of the suggested activities. The activity recommendation service 120 measures the estimated change in the physiological characteristic for each activity based on the difference in the physiological characteristic in the baseline projection compared to the corresponding estimated in each activity projection corresponding to the suggested activities. In addition to generating estimates of the average blood glucose levels for the PwD 104, which are correlated to the HbA1c levels, the virtual physiological model service 160 also generates estimates of changes in body mass and other physiological characteristics of interest in assisting the PwD 104 in managing diabetes.

The process 200 continues as the activity recommendation service 120 generates weight values based on the PwD preference data 140 (block 224). In general, each weight value is a numeric value in a predetermined range (e.g. 0.0 to 1.0 or any other suitable range) that corresponds to the likelihood of the PwD 104 performing a given activity on a consistent basis. For example, an activity that has a high likelihood of being performed may be assigned a higher numeric value that corresponds to a higher weight for the selected activity. In one configuration, the activity recommendation service 120 generates the numeric weight values from the preference data using an empirical weighting system based on posterior results of a large population of PwDs who have similar preferences to the PwD 104. For example, the activity recommendation service 120 uses a clustering algorithm or other suitable classification algorithm to find a group of PwDs with historical data stored in the preference data 140 of the memory 132 or an external database that represent a cohort of PwDs with similar preferences to the PwD 104. The activity recommendation service 120 then identifies the levels of adherence to different activities in the PwD physiological data 136 and other records for the cohort of PwDs, which provides a record with posterior results of how consistently PwDs with similar preferences to the PwD 104 actually perform different activities. In this example, the numeric weight values may be generated directly from the percentage of PwDs that successfully adhere to each activity based on the posterior data, although other numeric weighting systems may be used as well. The generation of the projections that is described above with reference to the processing of block 220 and the generation of the weight values based on the PwD preference data 140 may occur in any order or concurrently during the process 200.

The process 200 continues as the activity recommendation service 120 ranks the selected activities based on the estimated changes in the at least one physiological characteristic from the virtual physiological projection data 148 and the weight values corresponding to the user preference data 140 of the PwD 104 (block 228). The activity recommendation service 120 identifies the change in the at least one physiological characteristic by comparing the baseline projection that corresponds to the PwD baseline physiological profile to the corresponding activity projection of the activity physiological profile associated with each suggested activity. In one configuration, the activity recommendation service 120 generates a ranking by multiplication of the weight value by the numeric quantity of the change in the physiological characteristic for the activity relative to the baseline projection to generate a ranking score that accounts for both the potential benefit of performing the activity and the likelihood that the PwD 104 will actually perform the activity on a consistent basis. For example, consider activities A and B with regard to reducing body mass as a physiological characteristic. Activity A produces an estimated body mass reduction of 5 kg relative to the baseline projection with a user preference weight score of 0.7, while activity B produces an estimated body mass reduction of 7 kg relative to the baseline projection with a user preference weight score of 0.4. Using a multiplicative scaling factor, activity A has a ranking score of 0.7×5 kg=3.5 kg (scaled) while activity B has a ranking score of 0.4×7 kg=2.8 kg (scaled). In this example, activity A has the higher ranking score due to the scaling of the preference weight value even though the PwD 104 would be projected to have a greater weight loss if activity B were actually performed consistently. While the embodiment described above multiplies the weight value by a numeric value corresponding to the change in the physiological characteristic to generate a ranking score, other scaling operations may be used as well. For example, in another scaling configuration the preference weight score for an activity must exceed a predetermined threshold value to be considered for recommendation to the PwD 104. If multiple activities exceed the predetermined weight threshold, then the ranking algorithm ranks the remaining activities based on the greatest estimated improvement to the physiological characteristic to produce ranked results. Still other embodiments perform different scaling operations to rank the activities based on the estimated changes in the physiological characteristics from the virtual physiological model data 148 and the weight values from the preference data 140.

In some embodiments, the activity recommendation service 120 optionally filters any activities from the ranking process in which an estimated change in the physiological characteristic would produce an undesirable estimated outcome for the PwD 104. For example, if an activity is determined to reduce HbA1c beyond a maximum threshold that would be considered healthful for the PwD 104, then the activity recommendation service 120 filters the activity and will not generate a recommendation for the activity even if the process 200 produces a high ranking score for the activity. Similarly, the activity recommendation service 120 filters an activity that produces a reduction in body mass that is considered too large to be healthful for the PwD 104. The filtering process can also be applied to remove any activities that are estimated to produce worse results for the physiological characteristics of the PwD 104 relative to the estimate in the baseline projection, such as an undesirable increase in HbA1C or increase in body mass. While the physiological parameters for many PwDs may deteriorate from optimal levels over time as diabetes progresses even as the PwD 104 performs activities, the activity recommendation service 120 still filters activity recommendations with projections that produce worse estimated results than the baseline projection.

The process 200 continues as the system 100 generates an output that includes one or more of the suggested activities starting with the highest ranked activities identified as the one or more suggested activities that provide the greatest change in the physiological characteristic given the likelihood of the person with diabetes adhering to the activities (block 232). The suggested activities provide the basis for the coach 102 to consult with the PwD 104 to set goals for performing activities that the PwD has a high likelihood of performing in a consistent manner to achieve improvements in average blood glucose levels and body mass. In the configuration of FIG. 1, the activity recommendation service 120 transmits one or more of the potential activities to the coach terminal 112, the PwD electronic device 116, or both, which act as output devices that are operatively connected to the processor 124 in the activity recommendation service 120 via the network 118. In one configuration, the activity recommendation service 120 only generates an output with a single recommended activity that has the highest ranking. In another configuration, the activity recommendation service 120 generates an output that includes the highest ranked activity recommendations in two or more categories, such as generating an output with the highest ranked exercise activity and the highest ranked diet activity recommendation. In still another embodiment, the activity recommendation service 120 provides the output with multiple activity recommendations that are ranked from highest to lowest for the coach 102 and the PwD 104 to incorporate into a diabetes treatment plan. In addition to ranking the activities, the output of each ranked activity optionally includes the estimated change in the physiological characteristic from the virtual physiological model projection data 148 and the weight value or other metric that provides an estimate of how likely the PwD 104 is to adhere to the activity based on the PwD preference data 140.

While the embodiments described above for the system 100 and method 200 provide activity recommendations to the coach 102 and the PwD 104 as part of a coaching session, those of skill in the art will recognize that the PwD 104 may utilize the system 100 and the method 200 directly using the PwD electronic device 116. For example, in one embodiment the PwD 104 uses the PwD electronic device 116 to execute a web browser or other client program that accesses the system 100. The PwD electronic device 116 provides physiological data and preference data to the system 100 to enable the PwD 104 to receive the ranked recommendations for one or more activities from the system 100.

This disclosure is described in connection with what are considered to be the most practical and preferred embodiments. However, these embodiments are presented by way of illustration and are not intended to be limited to the disclosed embodiments. Accordingly, one of skill in the art will realize that this disclosure encompasses all modifications and alternative arrangements within the spirit and scope of the disclosure and as set forth in the following claims.

What is claimed is:

1. A method for generating activity recommendations in a diabetes treatment plan comprising:
receiving, with a processor, physiological data for a person with diabetes (PwD), preference data for the PwD, and a plurality of suggested activities for the PwD;
generating, with the processor, a plurality of physiological profiles for the PwD, the plurality of physiological profiles comprising:
a baseline physiological profile based on the physiological data for the PwD; and
a plurality of activity physiological profiles, each activity physiological profile corresponding to one activity in the plurality of suggested activities, and each activity physiological profile being based on the physiological data for the PwD and a modification of the physiological data associated with the one activity in the plurality of suggested activities corresponding to the activity physiological profile;
providing, with the processor, the plurality of physiological profiles to a virtual physiological model;
receiving, with the processor, a plurality of projections for the PwD from the virtual physiological model, each projection in the plurality of projections providing an estimated change in a physiological characteristic in the PwD during a predetermined time period corresponding to one physiological profile in the plurality of physiological profiles;
generating, with the processor, a plurality of weighted values based on the plurality of suggested activities and the preference data, each weighted value corresponding to a likelihood of the PwD adhering to a corresponding one of the suggested activities;
ranking, with the processor, each activity in the plurality of suggested activities based on the estimated change in the physiological characteristic of a projection in the plurality of projections associated with the activity relative to a baseline physiological projection in the plurality of projections corresponding to the baseline physiological profile and scaled by the weighted value corresponding to each activity; and
generating, with the processor, an output including a predetermined number of the plurality of suggested activities in order based on the ranking to identify one or more suggested activities that provide a greatest change in the physiological characteristic given the likelihood of the PwD adhering to the suggested activities.

2. The method of claim 1, the plurality of suggested activities further comprising:
at least one suggested exercise, at least one suggested change in diet, at least one suggested change in sleep hygiene, or at least one recommendation for medication adherence.

3. The method of claim 1, the preference data further comprising:
numeric data corresponding to answers to predetermined survey questions received from the PwD;
geographic data corresponding to a home location of the PwD; and
demographic information of the PwD.

4. The method of claim 1, wherein the estimated change in the physiological characteristic is an estimated change in a percentage of hemoglobin A1c (HbA1c) in blood of the PwD.

5. The method of claim 4 further comprising:
filtering, with the processor, an activity in the plurality of suggested activities to prevent the activity from being generated in the output in response to the estimated reduction in HbA1c for the activity exceeding a maximum HbA1c reduction threshold for the PwD.

6. The method of claim 1, wherein the estimated change in the physiological characteristic is an estimated change in body mass.

7. The method of claim 1, wherein the plurality of suggested activities for the PwD are received via a network from at least one of a terminal of a coach or an electronic device of the PwD.

8. The method of claim 1, wherein the plurality of suggested activities for the PwD are received from a database storing all recognized suggested activities.

9. The method of claim 1, wherein the preference data for the PwD are received via a network from at least one of a terminal of a coach or an electronic device of the PwD.

10. The method of claim 1, wherein the predetermined time period is one of 30 days, three months, six months, or a year.

11. A system for generating activity recommendations comprising:
a memory;
a network interface device; and
a processor operatively connected to the memory and the network interface device, the processor configured to:
store physiological data for a person with diabetes (PwD), preference data for the PwD, and a plurality of suggested activities for the PwD in the memory;
generate a plurality of physiological profiles for the PwD, the plurality of physiological profiles comprising:
a baseline physiological profile based on the physiological data for the PwD; and
a plurality of activity physiological profiles, each activity physiological profile corresponding to one activity in the plurality of suggested activities, and each activity physiological profile being based on the physiological data for the PwD and a modification of the physiological data associated with the one activity in the plurality of suggested activities corresponding to the activity physiological profile;
transmit, with the network interface device, the plurality of physiological profiles to a virtual physiological model service;
receive, with the network interface device, a plurality of projections for the PwD from the virtual physiological model service, each projection in the plurality of projections providing an estimated change in a physiological characteristic in the PwD during a predetermined time period corresponding to one physiological profile in the plurality of physiological profiles;
generate a plurality of weighted values based on the plurality of suggested activities and the preference data, each weighted value corresponding to a likelihood of the PwD adhering to a corresponding one of the suggested activities;
rank each activity in the plurality of suggested activities based on the estimated change in the physiological characteristic of a projection in the plurality of projections associated with the activity relative to a baseline physiological projection in the plurality of projections corresponding to the baseline physiological profile and scaled by the weighted value corresponding to each activity; and
generate an output including a predetermined number of the plurality of suggested activities in order based on the rank to identify one or more suggested activities that provide a greatest change in the physiological characteristic given the likelihood of the PwD adhering to the suggested activities.

12. The system of claim 11, the processor being further configured to:
transmit, with the network interface device, the output to at least one of a terminal of a coach or an electronic device of the PwD.

13. The system of claim 11, the plurality of suggested activities further comprising:
at least one suggested exercise, at least one suggested change in diet, at least one suggested change in sleep hygiene, or at least one recommendation for medication adherence.

14. The system of claim 11, the preference data further comprising:
numeric data corresponding to answers to predetermined survey questions received from the PwD;
geographic data corresponding to a home location of the PwD; and
demographic information of the PwD.

15. The system of claim 11, wherein the estimated change in the physiological characteristic is an estimated change in a percentage of hemoglobin A1c (HbA1c) in blood of the PwD.

16. The system of claim 15, the processor being further configured to:
filter an activity in the plurality of suggested activities to prevent the activity from being generated in the output in response to the estimated reduction in HbA1 c for the activity exceeding a maximum HbA1c reduction threshold for the PwD.

17. The system of claim 11, wherein the estimated change in the physiological characteristic is an estimated change in body mass.

18. The system of claim 11, wherein the plurality of suggested activities for the PwD are received via a network from at least one of a terminal of a coach or an electronic device of the PwD.

19. The system of claim 11, the memory being further configured to:
store a database of all recognized suggested activities for the PwD. The system of claim 11, wherein the preference data for the PwD are received via a network from at least one of a terminal of a coach or an electronic device of the PwD.

21. The system of claim 11 wherein the predetermined time period is one of 30 days, three months, six months, or a year.
